(19) United States
(12) Patent Application Publication
(10) Pub. No.: US 2005/0093974 A1
(43) Pub. Date: May 5, 2005

(54) Endoscope processor

(75) Inventors: Haruhiko Hibi, Tokyo (JP); Takuma Ohtaki, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 10/979,201

(22) Filed: Nov. 3, 2004

(30) Foreign Application Priority Data
Nov. 4, 2003 (JP) 2003-373967

(51) Int. Cl. H04N 7/18; A61B 1/04

(52) U.S. Cl. 348/71; 348/65

(57) ABSTRACT

A processor, to which an endoscope is connectable, for processing signals generated by the endoscope into image data, the processor including a light source which supplies light to the endoscope connected to the processor to illuminate a target area via the endoscope so that the endoscope can capture an image of the illuminated target area and convert the image into the signals, and a memory. The processor is configured so that data stored in the memory can be rewritten from outside the processor via an external connection device connected to the processor.

### FIELD OF THE INVENTION

The present invention relates to a processor which processes signals generated by an endoscope connected thereto, and more specifically relates to such a processor whose maintainability is enhanced.

### DESCRIPTION OF THE RELATED ART

A typical endoscope system is generally provided with an endoscope (electronic endoscope), an endoscope processor (hereinafter referred to simply as a “processor”) and a monitor. The endoscope includes an image pick-up device, and is operated to obtain signals of images captured inside the human body via the image pick-up device, and the processor fosters the imaging operation of the endoscope and transforms the signals output from the image pick-up device into image data which is displayed as a visual image on the monitor. In endoscope systems in recent years, a printer for printing image signals obtained by the processor, a personal computer for controlling various processing operations performed by the processor, and an external connection device such as a keyboard designed specially for the endoscope system to input various kinds of data to the processor are connected to the processor.

In such conventional endoscope systems, the processor incorporates a CPU, various circuits (e.g., a signal-processing circuit), a light source for the endoscope and a memory in which information necessary for operations of the CPU and the various circuits is stored. However, no conventional endoscope system is designed so that an external connection device such as a personal computer which is connected to the processor can obtain access to the memory. In addition, no conventional endoscope system is designed so that image data and historical imaging data thereof, which are obtained from image signals captured by the endoscope, are stored in the memory, rather, conventional endoscope systems are designed so that moving images contained in the obtained image data are displayed on the monitor while still images contained in the obtained image data are either printed on a printer in accordance with the still-image data output from the processor or stored in a memory of a personal computer or an external memory.

A technique of storing data of an endoscope connected to a processor in an internal memory integrated into the processor in an electronic endoscope system is disclosed in Japanese unexamined patent publication 2003-132151. This technique makes it possible to reliably record data of an endoscope in use which is necessary to be recorded. However, with such a technique, data other than that of the endoscope such as data of the processor itself, the image data and historical imaging data are not recorded in addition to the data of the endoscope in use.

In the processor incorporated in such an endoscope system disclosed in the aforementioned patent publication, an external connection device such as a personal computer which is connected to the processor cannot obtain access to the memory as noted above. Therefore, when maintenance is performed on the processor, data on use of the processor which is necessary for the maintenance thereof cannot be obtained from the processor. On this account, every time maintenance is performed on the processor, the processor inputs or records the historical imaging data into a personal computer or the like. Additionally, when any still image obtained in the past needs to be checked for maintenance of the processor, the still image must be read out of a personal computer or an external device which was connected to the processor before. Accordingly, it is difficult to carry out maintenance on the processor solely with the processor; maintenance of the processor needs to be carried out at a site where the processor is set up even with an external connection device such as a computer being connected to the processor, which makes operations for the maintenance complicated and troublesome.

The aforementioned patent publication does not disclose any system which accesses a memory provided in the processor from an external connection device (e.g., a personal computer) connected to the processor to write or read out various kinds of data into and from the memory. Therefore, an external connection device such as a personal computer cannot obtain access even to a processor ROM provided inside the processor. Accordingly, if a software program written in the processor ROM is to be upgraded in order to improve one or more capabilities of the processor, the user needs to take the trouble to go to the installation site of the processor to replace the processor ROM in which the software program to be upgraded is stored.

### SUMMARY OF THE INVENTION

The present invention provides a processor connectable to an endoscope to process signals generated by the endoscope, wherein a memory provided in the processor is made to be accessible from outside the processor so that data stored in the memory can be rewritten, thereby enhancing maintainability of the processor.

According to an aspect of the present invention, a processor, to which an endoscope is connectable, is provided for processing image signals generated by the endoscope into image data, the processor including a light source which supplies light to the endoscope connected to the processor to illuminate a target area via the endoscope so that the endoscope can capture an image of the illuminated target area and convert the image into the image signals, and a memory. The processor is configured so that data stored in the memory can be rewritten from outside the processor via an external connection device connected to the processor.

It is desirable for the memory to include a processor ROM in which a software program for controlling operations of the processor is stored, and for the external connection device to have access to the processor ROM so that the software program can be rewritten via the external connection device.

It is desirable for the external connection device to include a personal computer connected to the processor, so that the software program can be rewritten via the personal computer.

It is desirable for the memory to include an internal memory in which historical imaging data associated with still-image data obtained from the image data can be stored.

The still-image data and the historical imaging data which is obtained at the time the still-image data is captured can be stored in the internal memory to correspond to each other.

It is desirable for the external connection device to have access to each of the processor ROM and the internal memory.

It is desirable for the historical imaging data to include at least data on a patient examined with the endoscope.

The still-image data and the historical imaging data, which are stored in the internal memory, can be read out from the internal memory in response to a request from the external connection device.

According to the present invention, since the software program stored in the memory can be rewritten to be upgraded via an external connection device (e.g., a personal computer) connected to the processor directly or indirectly via a LAN, the memory does not have to be replaced to upgrade the software program stored in the memory; specifically, the user no longer needs to take the trouble to go to the installation site of the processor to replace the memory including the software program which is to be upgraded, thereby maintainability of the processor can be enhanced.

The present disclosure relates to subject matter contained in Japanese Patent Application No.2003-373967 (filed on Nov. 4, 2003) which is expressly incorporated herein by reference in its entirety.

### BRIEF DESCRIPTION OF THE DRAWINGS

The present invention will be described below in detail with reference to the accompanying drawings in which:

FIG. 1 is a diagrammatic representation of an endoscope system according to the present invention;

FIG. 2 is a schematic block diagram of the endoscope system shown in FIG. 1;

FIG. 3 is a flow chart of a main routine MAIN of a processor shown in FIG. 1;

FIG. 4 is a flow chart of a subroutine MEMORY STORAGE NUMBER ASSIGNMENT called from the main routine of in FIG. 3;

FIG. 5 is a flow chart showing a subroutine IMAGE RECORD called from the main routine of FIG. 3;

FIG. 6 is an explanatory format of the data stored in an internal memory provided in the processor;

FIG. 7 is a flow chart showing a subroutine DATA READOUT REQUEST called in the main routine shown in FIG. 3; and

FIG. 8 is a flow chart showing an interrupt routine VERSION UPGRADE.

### DESCRIPTION OF THE PREFERRED EMBODIMENT

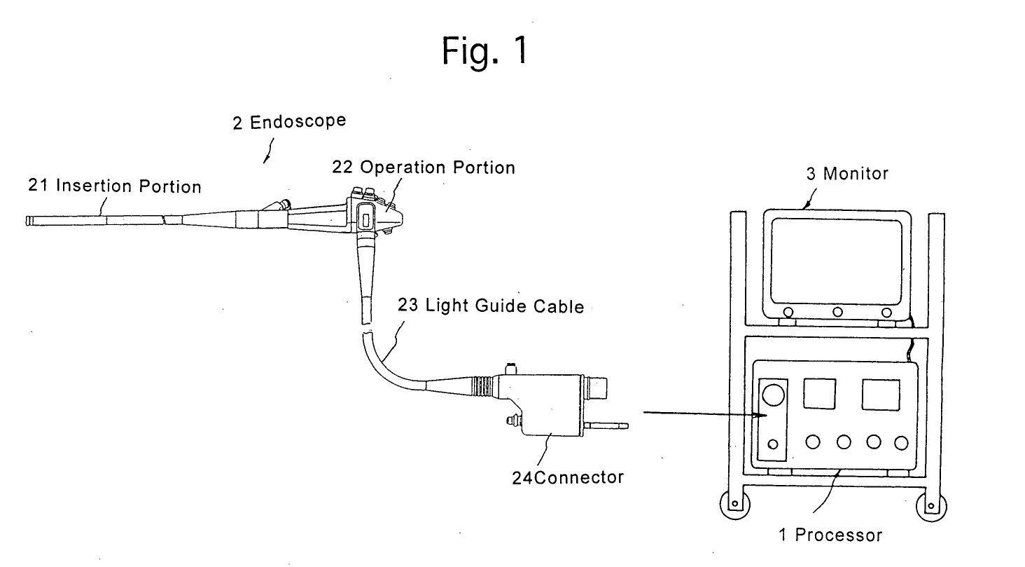

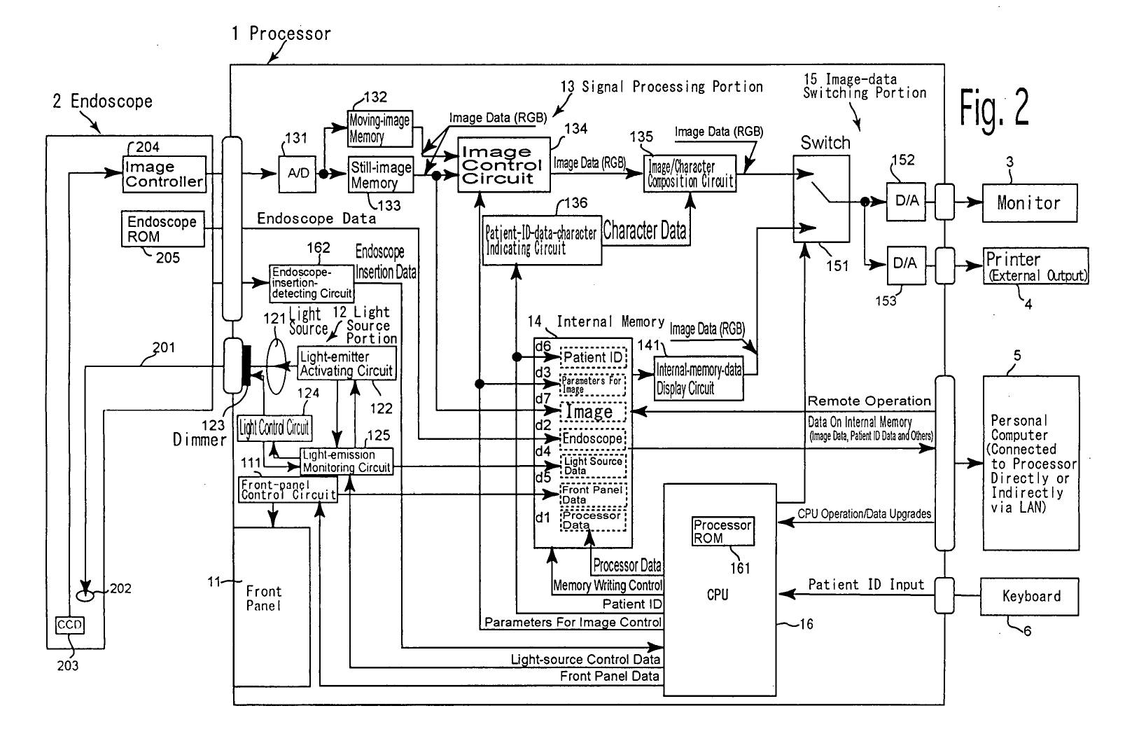

An embodiment of an endoscope system according to the present invention will be hereinafter discussed with reference to FIG. 1. FIG. 1 is a diagrammatic representation of an endoscope system using a processor (endoscope processor) 1 according to the present invention. The endoscope system is provided with a processor 1, an endoscope (electronic endoscope) 2 and a monitor 3. FIG. 2 is a schematic block diagram of the endoscope system shown in FIG. 1. The endoscope 2 is detachably connected to the processor 1. Image signals captured by the endoscope 2 are processed in the processor 1 to be output as video signals so that the video signals are displayed on a monitor 3 connected to the processor 1 or printed as printed images on a printer (video printer) 4 connected to the processor 1. The endoscope system is provided with a personal computer (external connection device) 5 and a keyboard (external connection device) 6 which serves as an input device for inputting data such as patient ID data to the processor 1. As will be discussed later, various kinds of data are output from the processor 1 to be input into the personal computer 5, or the keyboard 6 is operated to input data such as patient ID data into the processor 1.

As shown in FIG. 1, the endoscope 2 is provided with an insertion portion (insertion tube) 21, an operation portion 22, a light guide cable 23 and a connector 24. The insertion portion 21 is inserted into the body of a patient. The connector 24 is detachably connected to the processor 1. As shown in FIG. 2, the endoscope 2 is provided inside thereof with a light guide 201 which is arranged to pass through the light guide cable 23, the operation portion 22 and the insertion portion 21. The light emitted from a light source (lamp) 121 provided in the processor 1 is transmitted through the light guide 201 to illuminate the area in front of the tip end of the insertion portion 21. A CCD image sensor 203 and a light distribution lens 202 are provided at the distal end portion of the inserting portion 21. The light emitted from the light source 121 and passed through the light guide 201 is diffused through the light distribution lens 202 to illuminate a wide area inside a body cavity, and this illuminated wide area is photographed by the CCD image sensor 203. Image signals generated by the CCD image sensor 203 upon the illuminated area being photographed by the CCD image sensor 203 are output to the processor 1 via an image controller 204 provided in the endoscope 2. The endoscope 2 is provided therein with an electrically erasable/programmable endoscope ROM (EEPROM) 205 into which data such as endoscope data (which will be discussed later) is stored. This endoscope data can be output to the processor 1 upon the endoscope 2 being connected to the processor 1. The operation of the endoscope 2 is the same as that of a conventional endoscope, and will not be hereinafter discussed in detail. Image signals captured by the endoscope 2 are processed through a signal processing portion 13 provided in the processor 1 so that moving images and still images are displayed on the monitor 3 in accordance with obtained moving-image data and still-image data, respectively.

The processor 1 is provided with a front panel 11, a light source portion 12, the aforementioned signal processing portion 13, an internal memory 14 and a image-data switching portion 15, respective operations of which are controlled by a CPU 16 provided in the processor 1. Regarding the front panel 11, a front-panel control circuit 111 which is provided in the processor 1 and controlled by the CPU 16 controls the operation of the front panel 11 so that the front panel 11 visually indicates a variety of information related to the front panel 11, or captures front-panel data input by an operation of the front panel 11 so that the captured front-panel data is stored in the internal memory 14 by the CPU 16.

The light source portion 12 is provided with the light source 121, a light-emitter activating circuit 122, a dimmer 123, a light control circuit 124 and a light-emission monitoring circuit 125. The light-emitter activating circuit 122 operates to activate the light source 121. The dimmer 123 controls the intensity of the light source 121. The light-emission monitoring circuit 125 monitors whether the light source 121 is lighting at a desired intensity set by the dimmer 123. The CPU 16 controls the intensity of the light source 121 in accordance with the results of monitoring by the light-emission monitoring circuit 125 so that the light source 121 stays on at a desired intensity set by the dimmer 123. Illumination light emitted by the light source 121 is controlled to have a desired intensity via the dimmer 123, and is subsequently supplied to the endoscope 2 to illuminate the area in front of the end of the insertion portion 21 via the light distribution lens 202 and the light guide 201. Light-source data obtained in the light-emission monitoring circuit 125 is stored in the internal memory 14 by the CPU 16.

The signal processing portion 13 converts analogue image signals, which are generated by the CCD image sensor 203 of the endoscope 2 to be output to the processor 1, into digital image signals. The signal processing portion 13 also performs various kinds of signal processes for producing image signals in indicating visual images on the monitor 3. Image signals input from the endoscope 2 are converted into digital signals by an A/D converter 131 to be stored in a moving-image memory 132, which serves as a frame memory for moving images, and a still-image memory 133, which serves as a memory for still images which are captured as needed, as appropriate at the correct time. Digital image signals stored in each of the moving-image memory 132 and the still-image memory 133 are translated to desired image data via an image control circuit 134 to be input to an image/character composition circuit 135. In the image/character composition circuit 135, the input image data is combined with character data output from a patient-ID-data-character indicating circuit 136 to be output as image data combined with characters from the image/character composition circuit 135. The patient-ID-data-character indicating circuit 136 creates characters which correspond to the patient ID that is input from the keyboard 6 connected to the processor 1, and outputs character data on the created characters to the image/character composition circuit 135. It is possible for the image control circuit 134 to generate data corresponding to a combination of moving images and a still image via an operation of the front panel 11, and for the moving images and still images area to be indicated on the monitor 3.

The image-data switching portion 15 is provided with a switch 151 and two D/A converters: a first D/A converter 152 and a second D/A converter 153. The CPU 16 commands the switch 151 to selectively switch between the image data output from the image/character composition circuit 135 and the image data read out of the internal memory 14 by an internal-memory-data display circuit 141 which is connected to the internal memory 14. Each of the two D/A converters 152 and 153 converts the input image data that is selected by the switch 151 into analog signals, which are in turn output to the monitor 3 and the printer 4 to be capable of being displayed on the monitor 3 and printed on the printer 4, respectively.

The personal computer 5 and the processor 1 can be connected to each other via a local area network (LAN). For example, the processor 1 can be remote accessed via a LAN from a personal computer 5 provided in another room such as a server room.

The CPU 16 is provided therein with a processor ROM (EEPROM) 161 in which various kinds of data such as programmed software for controlling the operation of CPU 16 are stored. The internal memory 14 and the processor ROM 161 are collectively referred to as a memory in the specific embodiment of the present invention described herein. The processor ROM 161 is not limited solely to a type of processor ROM which is integrated into a CPU chip of the CPU 16. Namely, the processor ROM 161 can be of a type independent of the CPU chip. The CPU 16 performs necessary operations in accordance with endoscope insertion data from an endoscope-insertion-detecting circuit 162 which is connected to the CPU 16.

The internal memory 14 is constructed as a flash memory or an EEPROM, to which the CPU 16 can write and read out each item of data. The following data (d1 through d7) can be written in and read out of the internal memory 14:
d1: Processor data (including model name and serial number of the processor, repair information, owner information, hardware assembly number and software version number)
d2: Endoscope data (including model name and serial number of the endoscope, repair information, owner information, and signal processing data)
d3: Image parameters (image color information)
d4: Light-source data (service life of lamp and the number of light-ups of lamp)
d5: Front-panel data (front-panel operational status)
d6: Patient ID (including name of patient and patient's ID number)
d7: Image data (still-image data)

Each of these items of data d1 through d7 written in the internal memory 14 can be read out of the internal memory 14 by the personal computer 5 to be displayed thereon. Conversely, each of these items of data d1 through d7 can be written into the internal memory 14 from the personal computer 5. A patient ID input via the keyboard 6 can be written into the internal memory 14 as data d6.

Although the personal computer 5 has access to the internal memory 14, the processor 1 is provided with a protective feature of prohibiting the personal computer 5 from having access to a programmed-software storing area in the processor ROM 161 of the CPU 16 during normal use. In the case that the personal computer 5 makes access to the processor ROM 161, for instance, the programmed-software storing area can simply be released from protection by operating the front panel 11.

A usage pattern of the above described endoscope system will be discussed hereinafter.

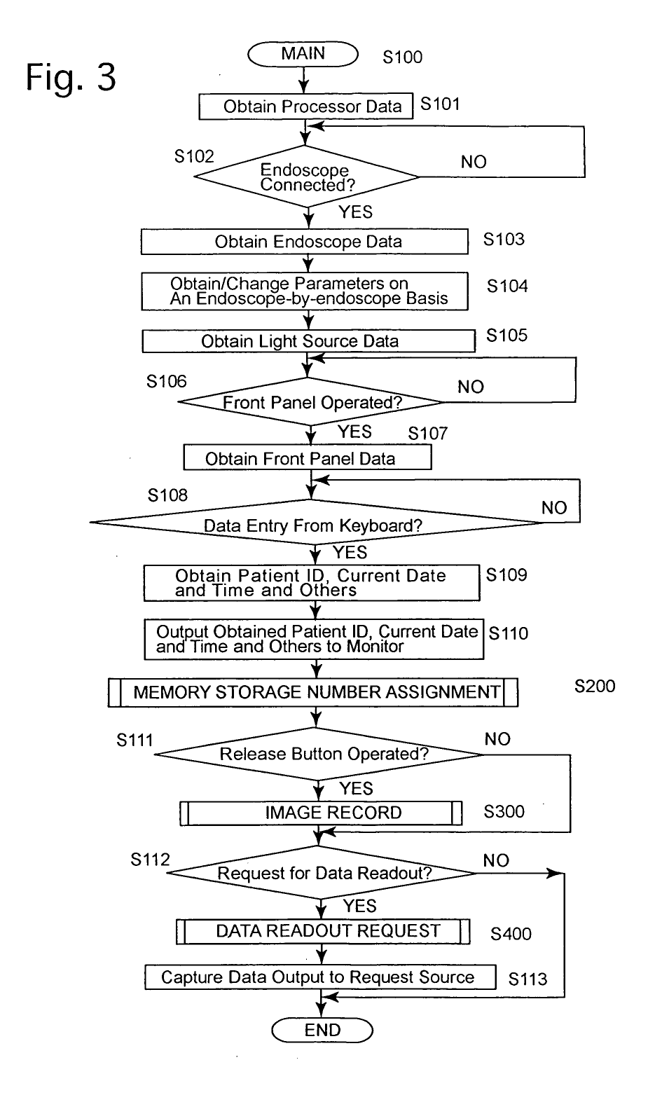

FIG. 3 is a flow chart showing a main routine MAIN (S100) of the processor 1 when the processor 1 starts up the endoscope system. Firstly, upon the power of the processor 1 being turned ON to start up the processor 1, the CPU 16 reads out and obtains processor data (e.g., model name and serial number of the processor) unique to the processor 1 which is prestored in the processor ROM 161 (step S101). Subsequently, it is determined whether the endoscope 2 is currently connected to the processor 1 (step S102). If the endoscope 2 is currently connected to the processor 1 (if YES at step S102), the processor 1 obtains the endoscope data from the endoscope ROM 205 (step S103). In addition, the processor 1 obtains image parameters which are to be set to correspond to image data that are obtained, together with the endoscope data, by an imaging operation of the endoscope 2 (step S104). Subsequently, the processor 1 obtains light source data from the light-emission monitoring circuit 125 of the light source portion 12 (step S105). Subsequently, it is determined whether the front panel 11 has been operated (control waits for the front panel 11 to be operated) (step S106). If it is determined that the front panel 11 has been operated (if YES at step S106), the CPU 16 obtains front panel data from the operated front panel 11 (step S107), and then it is determined whether data has been entered to the CPU 16 via the keyboard 6 (step S108). If data has been entered (if YES at step S108), the CPU 16 obtains the name of a patient, patient ID, current date and time, which are input in sequence from the keyboard 6 (step S109). Subsequently, the obtained items of information such as patient ID and current date and time are output to the monitor 3 to be displayed thereon as needed together with the image captured by the endoscope 2 (step S110).

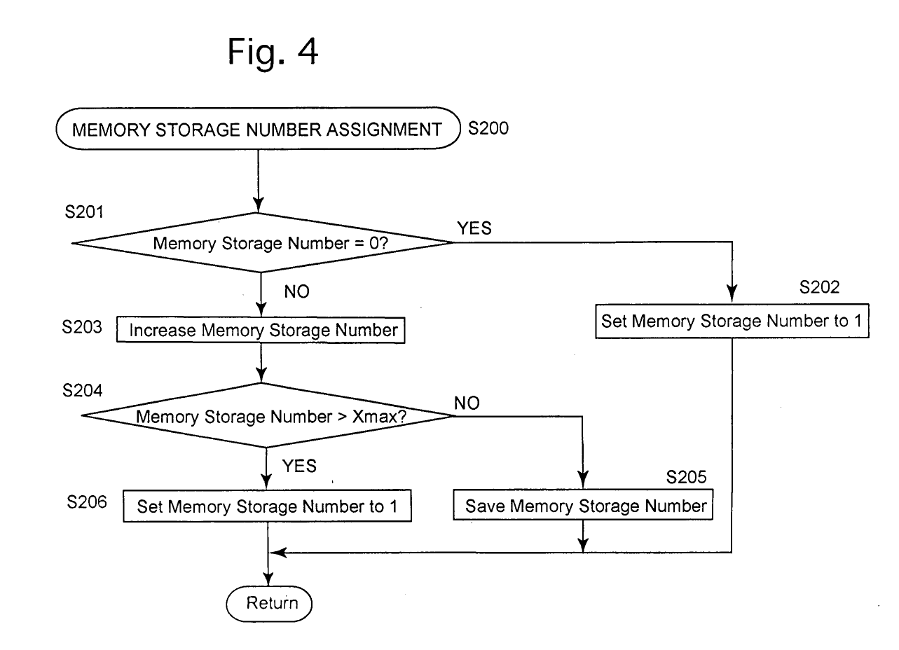

Immediately after the above described series of operations at steps S101 through S110 are completed, control enters a subroutine MEMORY STORAGE NUMBER ASSIGNMENT (step S200). In this subroutine, firstly it is determined whether the memory storage number is 0 (zero) as shown in FIG. 4 (step S201). If the memory storage number is zero (if YES at step S201), the memory storage number is set to 1 (step S202), otherwise (if NO at S201), the memory storage number is increased by one (step S203), and then it is determined whether the memory storage number is greater than a predetermined number Xmax (step S204). The number Xmax is predetermined to correspond to the maximum number of still images which can be stored in the internal memory 14. The maximum number of still images storable in the internal memory 14 is 36 in this particular embodiment, so that the number Xmax is equal to 36. If it is determined that the memory storage number is equal to or smaller than the number Xmax (if NO at S204), the memory storage number is saved (step S205), and control returns to the main routine. If it is determined that the memory storage number is greater than the number Xmax (if YES at step S204), the memory storage number is reset to 1 so that the first (oldest) still image stored in the internal memory 14 is overwritten with a new still image since thirty-six still images have been already stored in the internal memory 14 (step S206), and subsequently control returns to the main routine.

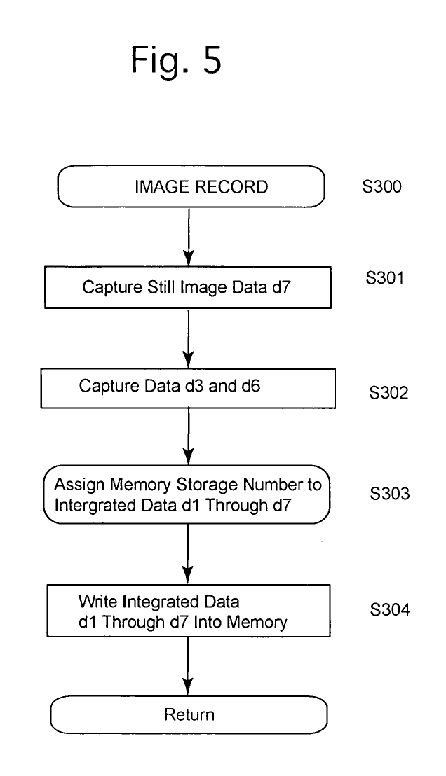

Upon completion of the subroutine MEMORY STORAGE NUMBER ASSIGNMENT at step S200, it is determined, from a signal output from the image controller 204 of the endoscope 2, whether a release button (not shown), provided on the endoscope 2, for capturing a still image of the illuminated area in front of the tip end of the insertion portion 21 of the endoscope 2 has been operated (step S111). If the release button has been operated (if YES at step S111), a subroutine IMAGE RECORD is performed (step S300). Referring to FIG. 5, immediately after an image is captured by the endoscope 2 with the endoscope 2 being coupled to the processor 1, signals generated by the CCD image sensor 203 are input to the processor 1 via the image controller 204 as shown in FIG. 2. In the processor 1, image data is produced from the signals generated by the CCD image sensor 203 via the signal processing portion 13, and is combined with character data output from the patient-ID-data-character indicating circuit 136 to be output as image data combined with characters from the image/character composition circuit 135 to be input to the image-data switching portion 15. In the image-data switching portion 15, the image data which is output to the monitor 3 to be displayed thereon via the switch 151 is switched to the aforementioned character-combined image data by the switch 151 so that the character-combined image data is displayed on the monitor 3. The still images displayed on the monitor 3 can be printed on the printer 4 as needed. While the combined image data is displayed on the monitor 3, still-image data d7 is extracted from the obtained image data to be captured in the still-image memory 133 (step S301). Subsequently, the data d3 and d6 (including character data to be combined with the still-image data d7) among the historical imaging data d1 through d6 that are obtained at steps S101 through S110 and the still-image data d7 are captured collectively as integrated data (step S302). The memory storage number obtained through the subroutine MEMORY STORAGE NUMBER ASSIGNMENT at step S200 is assigned to this integrated data (step S303), and then this integrated data (including data d3 through d7) is written into the internal memory 14 (step S304). Subsequently, control returns to the main routine.

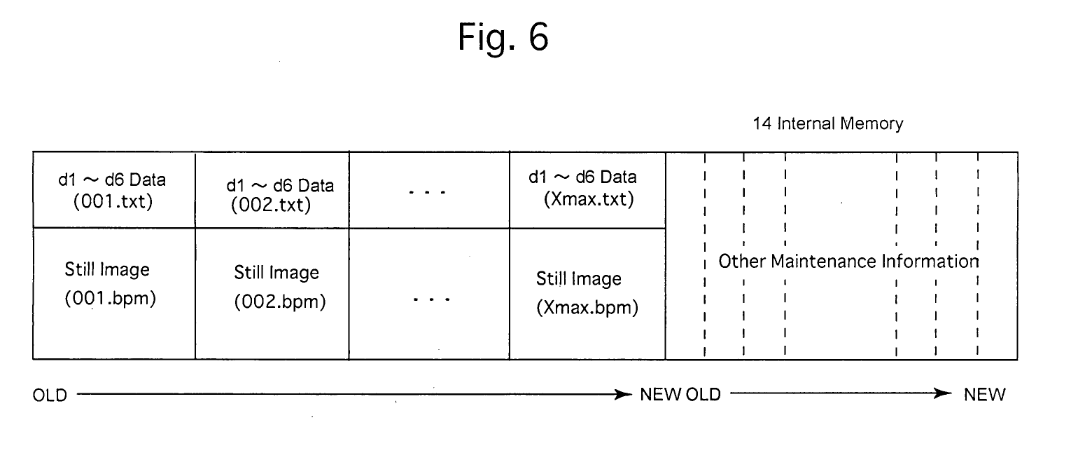

In this manner, two or more of such integrated data, each of which is made by combining the still-image data d7 with the associated necessary data of historical imaging data d1 through d6, are stored in the internal memory 14 while the memory storage number is increased one by one. The integrated data is repeatedly stored in the internal memory 14 until the number of still images reaches the number Xmax as shown in FIG. 6. When the number of stored still images exceeds the number Xmax, the oldest still-image data (data numbered 1) is erased and rewritten as new still-image data one by one as described in the descriptions of the subroutine MEMORY STORAGE NUMBER ASSIGNMENT at step S200. Each data d1 through d6 is stored in the internal memory 14 while the aforementioned memory storage number is assigned to the data, and also each data d1 through d6 is overwritten with new data d1 through d6 in order from oldest data, respectively, while the oldest still-image data is erased and rewritten as new still-image data one by one.

If the release button is not ON (step S111: NO), control skips step S300 and advances to step S112.

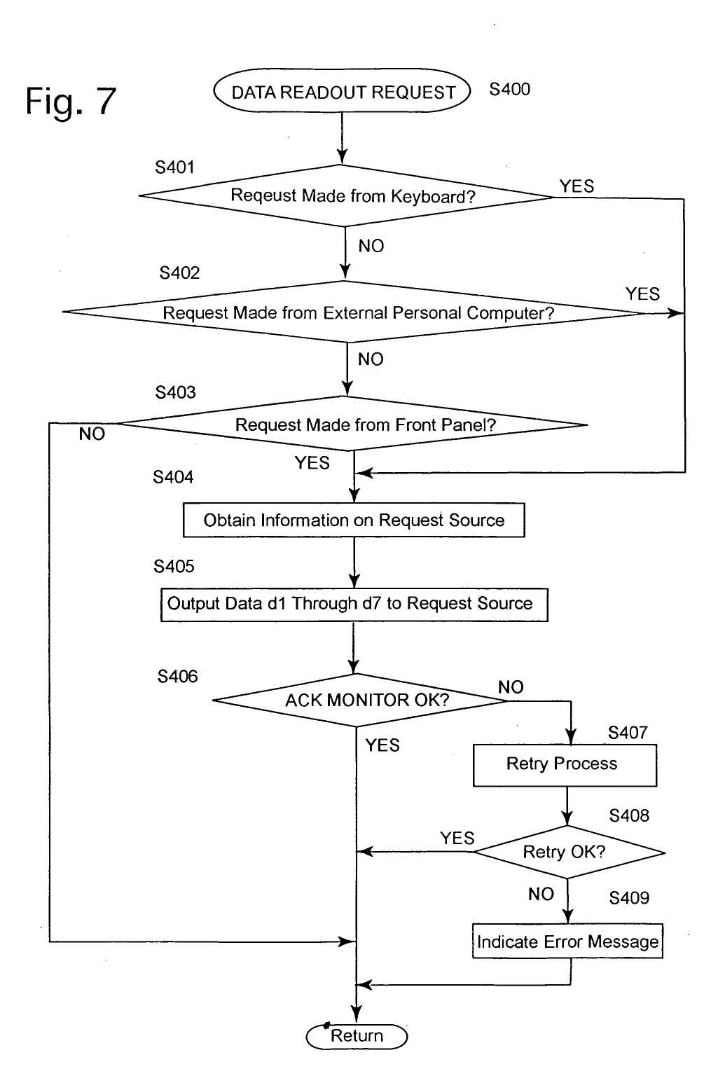

If there is a request for a readout of the data stored in the internal memory 14 for, e.g., maintenance on the processor 1, after each data d1 through d7 has been written into the internal memory 14 (step S112), a subroutine DATA READOUT REQUEST for searching a source of the request is performed (step S400), as shown in FIG. 7. In the subroutine DATA READOUT REQUEST, firstly it is determined whether the request has been made from the keyboard 6 (step S401), it is determined whether the request has been made from the external personal computer 5 (step S402), and it is determined whether the request has been made from the front panel 11 (step S403). If it is determined that the request has been made from the keyboard 6, the personal computer 5 or the front panel 11 (if YES at step S401, S402 or S403), information on the request source is obtained (step S404). If it is determined that the request has been made from neither the keyboard 6, the personal computer 5 nor the front panel 11 (if NO at each of steps S401, S402 and S403), control returns to the main routine.

Upon the request source being specified by obtaining information on the request source at step S404, desired data stored in the internal memory 14 is output to the request source (step S405). It is possible to specify one data (datum) from among a plurality of items of data (representing a plurality of still images) stored in the internal memory 14 by specifying a type of data to the CPU 16 from the request source. At this time, if the request source requests a specific memory storage number, the data bearing this memory storage number which is stored in the internal memory 14 is output to the request source. If the request source is the personal computer 5 while the personal computer 5 is on a LAN, data stored in the internal memory 14 can be read out from all the personal computers on the LAN.

When data is output from the internal memory 14, the data is output to the request source in accordance with the information obtained at step S404. At this time, an operation ACK (acknowledge) MONITOR is performed (step S406). This operation is performed whether data communications are properly carried out between the request source and the processor 1 when data is read out of the internal memory 14. If it is determined that the data communications are properly carried out (if YES at step S406), control returns to the main routine. If it is determined that the data communications are not properly carried out (if NO at step S406), a retry process is performed (step S407) and it is determined whether the retry process is successful (step S408). If the retry process is successful (if YES at step S408), control returns to the main routine. If the retry process is not successful (if NO at step S408), an error message is displayed on the monitor 3 (step S409), and control returns to the main routine. In this case, this error information can be written into the internal memory 14 after the memory storage number obtained in the same manner as in the subroutine at S200 is assigned to the error information.

In the main routine S100, the data output to the request source is captured by the request source thereat (step S113), so that an associated operation is performed at the request source. Specifically, if the request source is the monitor 3, the switch 151 operates so that a still image and the historical imaging data thereof are displayed on the monitor 3 in accordance with data obtained from the internal memory 14 via the internal-memory-data display circuit 141. If the request source is the printer 4, the switch 151 operates in a similar manner so that a still image with the historical imaging data thereof are printed via the printer 4 in accordance with data obtained from the internal memory 14 via the internal-memory-data display circuit 141. If the request source is the personal computer 5, the same data obtained by the internal-memory-data display circuit 141 is recorded in the personal computer 5.

Once the process at step S113 has finished, or if NO at step S112, control returns to step S101, and steps S101 through S113 are repeated while the power of the processor 1 is ON.

Regarding the data output from the processor 1, the still-image data d7 that is stored in the internal memory 14 and the historical imaging data d1 through d6 that is stored in the internal memory 14 to be associated with the still-image data d7 can be output from the processor 1. This still image data can be stored in the internal memory 14 until the number of stored still images reaches a predetermined number (36 in this particular embodiment), and the oldest still-image data has been overwritten with new still-image data sequentially in order from the oldest data when the number of stored still images exceeds the predetermined number. In the case where the personal computer 5 is connected to the processor 1, the still-image data d7 and the associated historical imaging data d1 through d6 can be read out sequentially from the internal memory 14 of the processor 1 by the personal computer 5 so that stored still images and the historical imaging data thereof can be seen on the processor 1, thus being capable of being used effectively at maintenance of the processor 1 and the endoscope 2.

When the still-image data d7 and the historical imaging data d1 through d6 need to be retrieved from the processor 1, such still-image data and the historical imaging data thereof which are stored in the processor 1 can be viewed on a personal computer or a monitor which is different from that used when the still image data was captured, so long as the personal computer or the monitor can interface with the processor 1. Therefore, if only a monitor is simply connected to the processor 1 to be capable of displaying a still image thereon or if only a printer is simply connected to the processor 1 so that a still image can be printed thereby, the processor 1 can perform maintenance thereof on a standalone basis even if a state of the endoscope system in which the processor 1 is connected to a personal computer or a LAN is not maintained. This improves the maintainability of the processor 1.

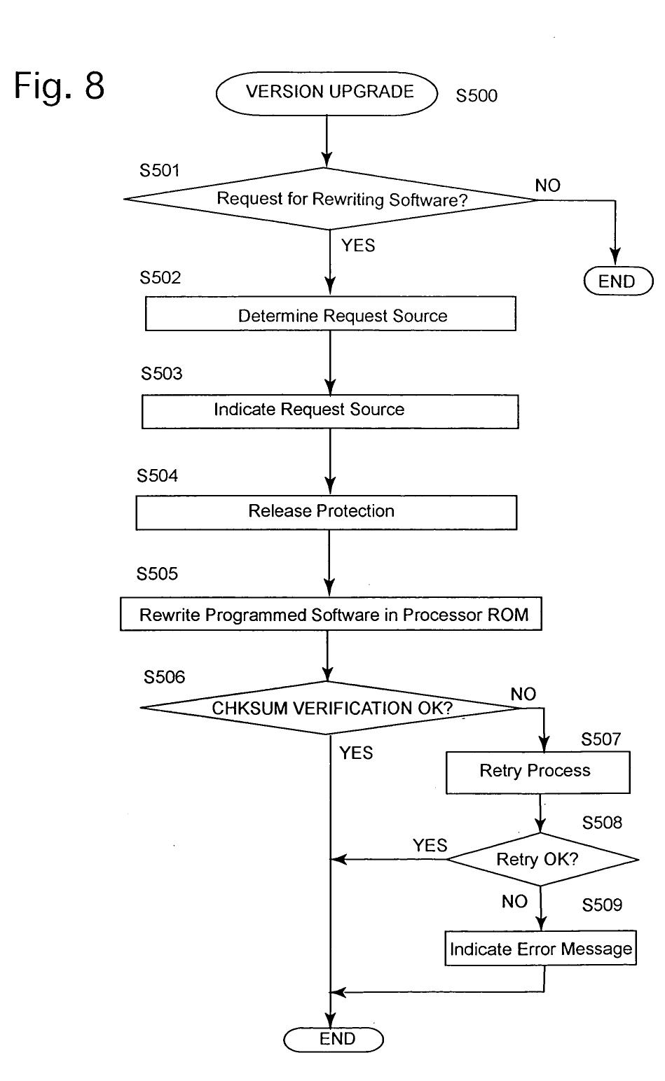

Software programs written in the processor ROM 161 in the CPU 16 or the endoscope ROM 205 in the endoscope 2 can be rewritten (reprogrammed) to be upgraded from a personal computer connected to the processor 1. FIG. 8 is a flow chart showing an interrupt routine VERSION UPGRADE. In this routine at S500, firstly it is determined whether there is a request for rewriting software (step S501). If YES at step S501, the memory-storage location of a software program which is to be rewritten is determined in accordance with a command for reprogramming from the personal computer (step S502). Assume here that it is determined at step S502 that a software program in the processor ROM 161 is to be rewritten, and the memory-storage location thereof is indicated on, e.g., the monitor 3 (step S503). At the same time, from this indication the user confirms the rewrite memory-storage location, and releases protection of the software program by operating the front panel 11 (step S504). Thereafter, a new software program is input to the processor 1 from the personal computer 5 via an operation of the personal computer 5, and is input to the processor ROM 161 so that the old software program is erased and rewritten as the new software program (step S505). When the old software program is rewritten to be replaced by the new software program, a software version number of the software program written in the processor ROM 161 is also rewritten to a new software version number. Upon completion of the rewriting operation at step S505, an operation CHKSUM VERIFICATION is performed (step S506). In this operation, it is determined whether data communication is properly carried out by checking an error checking signal included in the new software program. If it is determined that data communication has been properly carried (if YES at step S506), a rewrite completion message is displayed on, e.g., the monitor 3 and control ends. If data communication has not been properly carried (if NO at step S506), a retry process is performed (step S507) and it is determined whether the retry process is successful (step S508). If the retry process is successful (if OK at step S508), a rewrite completion message is displayed on, e.g., the monitor 3 and control ends. If the retry process is not successful (if NO at step S508), an error message is displayed on, e.g., the monitor 3 (step S509), and control ends.

If there is no request to rewrite software (NO at step S501), control ends.

Software programs and data of the endoscope 2 which are written in the endoscope ROM 205 in the endoscope 2 can be rewritten via the processor 1 in a manner similar to the above described manner of rewriting a software program written in the processor ROM 161 in the CPU 16. In this case, referring to the flow chart shown in FIG. 8, it is determined at step S502 that a software program in the endoscope ROM 205 is to be rewritten, and this result of determination is indicated on, e.g., the monitor 3 at step S503. From this indication the user confirms the memory-storage location which is to be rewritten, and releases protection of the software program stored in the endoscope ROM 205 at step S504. Thereafter, a software program stored in the endoscope ROM 205 is rewritten to be replaced by a new software program while a software version number of the software program written in the endoscope ROM 205 is also rewritten as a new software version number at step S505. Upon completion of the rewriting operation at step S505, an operation CHKSUM VERIFICATION (step S506) is performed.

As can be understood from the above descriptions, since the software program stored in the memory can be rewritten to be upgraded via an external connection device (e.g., a personal computer) connected to the processor directly or indirectly via a LAN, the user no longer needs to take the trouble to go to the installation site of the processor to replace either a CPU including the processor ROM or the processor ROM provided independently of a CPU which makes it possible to enhance maintainability of the processor.

Although a software program is written into a processor ROM integrated into a CPU in the above described embodiment of the processor, it is possible that an internal memory of the processor be partitioned to designate a part of the internal memory as a processor ROM into which the software program is written. In this case, the software program can be upgraded with an external connection device such as a personal computer through the same route as the case of reading and writing the image data and the historical imaging data, which is advantageous to simplify the operability of the endoscope system.

Obvious changes may be made in the specific embodiment of the present invention described herein, such modifications being within the spirit and scope of the invention claimed. It is indicated that all matter contained herein is illustrative and does not limit the scope of the present invention.

1. A processor, to which an endoscope is connectable, for processing image signals generated by said endoscope into image data, said processor comprising:
a light source which supplies light to said endoscope connected to said processor to illuminate a target area via said endoscope so that said endoscope can capture an image of said illuminated target area and convert said image into said image signals; and
a memory,
wherein said processor is configured so that data stored in said memory can be rewritten from outside said processor via an external connection device connected to said processor.

2. The processor according to claim 1, wherein said memory comprises a processor ROM in which a software program for controlling operations of said processor is stored, and
wherein said external connection device has access to said processor ROM so that said software program can be rewritten via said external connection device.

3. The processor according to claim 2, wherein said external connection device comprises a personal computer connected to said processor, so that said software program can be rewritten via said personal computer.

4. The processor according to claim 1, wherein said memory comprises an internal memory in which historical imaging data associated with still-image data obtained from said image data can be stored.

5. The processor according to claim 4, wherein said still-image data and said historical imaging data which is obtained at the time said still-image data is captured are stored in said internal memory to correspond to each other.

6. The processor according to claim 4, wherein said external connection device has access to each of said processor ROM and said internal memory.

7. The processor according to claim 4, wherein said historical imaging data includes at least data on a patient examined with said endoscope.

8. The processor according to claim 5, wherein said still-image data and said historical imaging data, which are stored in said internal memory, are read out from said internal memory in response to a request from said external connection device.
